(19) United States
(12) Patent Application Publication
(10) Pub. No.: US 2026/0007727 A1
(43) Pub. Date: Jan. 8, 2026

(54) DRUG THAT PROMOTES HEMOSTASIS

(71) Applicant: BIOMEDIZINISCHE FORSCHUNG & BIO-PRODUKTE GMBH, Wien (AT)

(72) Inventors: Johann Georg GRAUS, Wien (AT); Christoph MADER, Komeuburg (AT); Richard GÖLLES, Wien (AT); Michael PRÜCKLER, Wien (AT); Alexandru Michael-Eduard TREFILOV, Wien (AT); Marianne KUNSCHAK, Klosterneuburg (AT); Johann EIBL, Wien (AT)

(21) Appl. No.: 18/993,468

(22) PCT Filed: Jun. 23, 2023
(86) PCT No.: PCT/EP2023/067193

§ 371 Date: Jan. 10, 2025

(30) Foreign Application Priority Data
Jul. 12, 2022 (EP) 22184262.8

(51) Int. Cl. A61K 38/48; A61P 7/04

(52) U.S. Cl. CPC A61K 38/4833; A61P 7/04; C12Y 304/21005

(57) ABSTRACT

The invention relates to the use of prethrombin-1 as a drug.

The invention relates to a drug for promoting hemostasis and for treating hemorrhage.

Hemostasis is the result of a cascade-like, proteolytic activation of inactive zymogens. The concerted interaction of activated coagulation factors with their cofactors causes, as the penultimate stage of hemostasis, the activation of prothrombin to thrombin, which, if present in sufficient concentration, causes the fibrinogen present in the blood plasma to coagulate in the area of the injury and forms an insoluble fibrin matrix there together with activated thrombocytes.

The formation of thrombin is therefore a key element of hemostasis, with the generation of thrombin from prothrombin being a complex and strictly regulated enzymatic process. Other coagulation factors and cofactors are required for the entire hemostatic process, just like thrombocytes and certain endothelial factors.

The enzymatic activation of prothrombin occurs through factor Xa, an enzyme emerging through the activation of factor X. The activity of factor Xa is increased by several orders of magnitude when the enzyme complex prothrombinase is formed together with factor Va as a cofactor. Prothrombinase arises in the presence of calcium ions on phospholipid-containing membrane surfaces, which are formed by activated platelets or by the endothelium.

Factor Xa is formed by two different tenases: the intrinsic and the extrinsic tenases. The intrinsic tenase is characterized by the enzyme factor IXa and its cofactor VIIIa. The extrinsic tenase, in turn, constitutes the beginning of the coagulation cascade and consists of the enzyme factor VIIa and tissue factor, a tissue factor that comes into contact with blood when the endothelium is injured. The factor Xa formed by the extrinsic tenase is rapidly inhibited by TFPI. The tiny amounts of thrombin formed by the extrinsically produced factor Xa activate cofactors V and VIII as well as platelets at the site of the coagulation process (1).

Factor V or a shortened form of factor V (factor V short) are cofactors of TFPI besides protein S. When factor V or, respectively, factor V short is activated by thrombin, a decrease in the activity of the cofactor effect and hence a decrease in the inhibitory effect of TFPI occur (2).

Hemophilia A and B are pathological changes characterized by a dysfunction of the components of the intrinsic tenase. The malfunctions of the intrinsic tenase lead to disruptions in the hemostasis. The treatment of those coagulation disorders is done by substituting the missing coagulation factors. A common complication of this substitution treatment is the formation of alloantibodies against the substituted coagulation factor. These inhibitors hamper any further substitution or even render it completely ineffective.

Circumventing these inhibitors constitutes an important challenge in the treatment of hemophilia. In the early 1970s, prothrombin complexes were developed as drugs for the treatment of hemorrhage from a variety of causes. Prothrombin complexes also proved to be quite effective in case of inhibitor hemophilia. A further development consisted in activated prothrombin complexes, which increasingly replaced non-activated prothrombin complexes in the treatment of inhibitor hemophilia (3, 4). Factor VIIa as a component of extrinsic tenase also proved to be a possible therapeutic option for inhibitor hemophilia.

Activated factors, especially factor Xa, in combination with prothrombin were postulated as effective components of activated prothrombin complex concentrates (5). In the blood, prothrombin has a relatively long half-life, which causes the prothrombin concentration to be increased to two to three times the normal value by repeated administration of activated prothrombin complexes.

It was realized early on that, in addition to the concentration of antithrombin, the concentration of prothrombin in the blood is the most important parameter for normal blood coagulation (6).

Highly purified prothrombin proved to be hemostatically effective in mouse models of hemophilia A and B (7). The prothrombin concentration in plasma is approximately 80-90 mg/l. The half-life is 48-70 hours. In order to significantly increase the prothrombin concentration in plasma, application of high quantities of prothrombin is necessary, but a long-lasting effect is also achievable due to the long half-life. Nevertheless, the use of prothrombin as a drug was not followed up on. A clinical study to evaluate the safety, toxicity and pharmacodynamics of recombinant human prothrombin was terminated prematurely (8).

Prothrombin is a vitamin K-dependent proenzyme and has a complex structure characterized by an N-terminal Gla domain and the Kringle 1 domain (fragment 1), the Kringle 2 domain (fragment 2) and the actual enzyme domain. For the formation of thrombin, prothrombin must be cleaved at two sites: R271 and R320. Cleavage at site R320 results in the formation of the enzymatically active meizothrombin. Subsequent cleavage at site R271 results in the release of thrombin. If the cleavage site R271 of prothrombin is cleaved first, the enzymatically inactive prethrombin-2 is formed, which, subsequently, can be converted into thrombin, again by cleavage at site R320.

In the presence of cofactor Va, the cleavage site R320 is cleaved preferentially, and meizothrombin emerges as an intermediate product. In the absence of factor Va, however, the cleavage site R271 is cleaved preferentially, and mainly prethrombin-2 emerges (9).

The main part of the thrombin necessary for blood coagulation is formed by the prothrombinase complex, consisting of factor Xa and its cofactor Va, on phospholipid-containing surfaces in a concerted mechanism (10). However, thrombin also plays a crucial role in the activation of thrombocytes and the formation of cofactors Va and VIIIa, which are important for the two tenases.

Prethrombin-1 is an enzymatically inactive cleavage product which arises due to the cleavage of fragment 1 from prothrombin by thrombin in a feedback reaction (11, 12). Both prethrombin-1 and prethrombin-2 lack the Gla domain required for the membrane's binding capacity and, therefore, they fail to be efficiently activated in vitro by prothrombinase bound to phospholipid vesicles (13). In the absence of factor Va or, respectively, phospholipids, free factor Xa activates prothrombin and prethrombin-1 at similar rates to form thrombin.

When prethrombin-1 is generated on activated thrombocyte surfaces, activation to form thrombin occurs rapidly (14).

The fragment 2 present in prethrombin-1 has a sequence that binds factor Va (15). In prethrombin-2, this sequence, which is located on kringle 2, is missing.

McDuffie et al. (16) used specific immunoassays to determine the concentrations of prothrombin, thrombin and prothrombin fragments in the plasma of normal individuals and of patients with suspected disseminated intravascular coagulation (DIC). From the results obtained, it was concluded that measurements of prethrombin-1 levels are useful neither for the diagnosis nor for the treatment management of DIC.

Owen et al. (17) examined the catabolism of prothrombin and its fragments in normal dogs. To determine whether prethrombin-1 could play a role as a degradation product of prothrombin, they infused dogs with radiolabelled prethrombin-1. Based on the identified half-lives and the plasma concentrations of prethrombin-1 determined in normal individuals, they discovered that prothrombin is not catabolized via the release of prethrombin-1.

Lanchantin et al. (18) realized early on that, in a coagulation test conducted with prothrombin-deficient plasma, the prothrombin activity is lost due to the effect of thrombin on prothrombin. The two cleavage products arising as a result of the effect of thrombin on prothrombin were ultimately termed prethrombin-1 and fragment 1. Prethrombin-1 is enzymatically inactive.

Heldebrandt et al. (19) reported that prothrombin and prethrombin-1 (intermediate 1) can be activated to form thrombin in a diluted aqueous buffer by factor Xa at approximately the same rate. However, due to the lack of fragment 1, prethrombin-1 cannot bind to phospholipid membranes and is therefore convertible by prothrombinase only at a much lower rate than prothrombin. This is also the reason why prethrombin-1 has hardly any activity in the coagulation test with prothrombin-deficient plasma. The test method is based on a coagulometric coagulation test in which all coagulation factors are present in the plasma except prothrombin. The activity of prothrombin is measured by adding calcium and thromboplastin. Prothrombin is the limiting factor, and the coagulation time is inversely proportional to the concentration of prothrombin.

Seegers et al. (20) and Baker et al. (21) have also shown that prethrombin-1 can be converted into thrombin only with difficulty when phospholipids, factor Xa, factor V and calcium ions are used as procoagulants.

Seegers et al. (22, 23) described a previously unknown procoagulant which they termed protein M. This protein accelerated the formation of thrombin in a five-component system consisting of a thrombin zymogen, factor Xa, factor V, phospholipids and calcium ions.

The topical application of thrombin for hemostasis is widely used clinically (24, 25).

Disorders of the coagulation system can lead to severe impairment of hemostasis, with the direct administration of thrombin in hemorrhagic diatheses not being a treatment option because of the high risk of fatal systemic coagulation.

It is therefore of great interest to be able to provide treatment options with suitable drugs, that normalize prolonged bleeding times, especially in hemophilia A or B in the presence of inhibitors.

The currently available therapeutic options essentially comprise activated prothrombin complexes (FEIBA®, Takeda) or activated factor VII (NovoSeven®, Novo Nordisk). Activated factor VII has a very short half-life and is therefore not considered for prophylactic use. Activated prothrombin complexes have a complex composition, with zymogens and traces of activated forms of the procoagulant coagulation factors II, VII, IX, X, anticoagulant factors such as protein C and TFPI and small quantities of cofactors FV, FVIII and protein S being present in a balanced ratio (26).

It is the object of the present invention to provide a drug for promoting hemostasis and for treating hemorrhage which contains a molecularly clearly defined active ingredient and has a composition that is easy to formulate. Furthermore, the drug is supposed to be an alternative to the drugs commonly used today for promoting hemostasis, such as activated prothrombin complexes and activated factor VIIa.

### DESCRIPTION OF THE INVENTION

This object is achieved by means of a drug containing prethrombin-1 as an active ingredient. Thus, the invention consists in the use of prethrombin-1 as a drug or, respectively, in the use of prethrombin-1 for the manufacture of a drug.

A preferred embodiment of the invention consists of the specific use of prethrombin-1 for promoting hemostasis or, respectively, in the use of prethrombin-1 for the manufacture of a drug for promoting hemostasis.

Further preferred embodiments of the invention are:
prethrombin-1 for use in the treatment of hemorrhage or, respectively, the use of prethrombin-1 for the manufacture of a drug for treating hemorrhage;
prethrombin-1 for use in the treatment of hemorrhage in patients with coagulation disorders or, respectively, the use of prethrombin-1 for the manufacture of a drug for treating hemorrhage in patients with coagulation disorders;
prethrombin-1 for use in the treatment of hemorrhage resulting from trauma or internal injuries or, respectively, the use of prethrombin-1 for the manufacture of a drug for treating hemorrhage resulting from trauma or internal injuries;
prethrombin-1 for use in the treatment of breakthrough bleeds under substitution treatments with coagulation factors, therapies mimicking factor VIII or gene therapies or, respectively, the use of prethrombin-1 for the manufacture of a drug for treating breakthrough bleeds under substitution treatments with coagulation factors, therapies mimicking factor VIII or gene therapies; and
prethrombin-1 for use in the reversal of anticoagulation by direct-acting anticoagulants administered orally or, respectively, the use of prethrombin-1 for the manufacture of a drug for reversing anticoagulation by direct-acting anticoagulants administered orally.

Furthermore, the invention also comprises a method
of treating patients to promote hemostasis,
of treating hemorrhage,
of treating coagulation disorders,
of treating hemorrhage resulting from trauma or internal injuries,
of treating breakthrough bleeds under substitution treatments with coagulation factors, therapies mimicking factor VIII or gene therapies, and
of reversing anticoagulation by direct-acting anticoagulants administered orally, which methods comprise administering an effective amount of prethrombin-1 to a patient.

As described in the following example, prethrombin-1 can be prepared in a simple manner from prothrombin by cleavage with thrombin and subsequent chromatographic purification. Prethrombin-1 can be formulated with a suitable pharmaceutical vehicle, sterilized by filtration and aseptically filled. Freeze-drying is a preferred pharmaceutical preparation.

In order to use prethrombin-1 for the manufacture of a drug from prothrombin, various methods of virus inactivation or virus elimination can be applied. Examples of this are known to the person skilled in the art.

As an alternative to the extraction from plasma, prethrombin-1 can also be prepared by suitable molecular-biological methods, in particular using recombinant DNA technology. An additional, special embodiment of the present invention thus consists of using recombinant prethrombin-1 instead of native prethrombin-1 for the above-mentioned indications.

Prothrombin complex concentrates cannot be prepared using recombinant DNA technology. Their composition is too complex. Prothrombin, as the procoagulant factor thereof predominantly taking effect, must be kept in balance by sufficient amounts of anticoagulant components. As these have a shorter half-life than prothrombin, an excess of prothrombin and undesirable thrombogenic effects associated therewith can occur.

Prethrombin-1 is defined by the protein sequence that results when fragment 1 is cleaved from prothrombin at position R155.

The dosage of prethrombin-1 is based on the cause of the hemorrhage and the severity of the hemorrhage, but preferably amounts to 100 units/kg body weight, with one unit of prethrombin-1 being defined as the amount corresponding molecularly to one unit of prothrombin. Lower dosages can also cause hemostasis to be promoted. Higher dosages of up to 300 units/kg can be effective if the hemorrhage cannot be stopped by standard dosages. Plasma levels can be increased further by repeatedly administering prethrombin-1.

It has been shown that prethrombin-1 has only a low tendency to cause undesirable thromboembolic events.

The efficacy of prethrombin-1 as a drug with hemostatic activity is shown below in an FVIII inhibitor mouse model. The inventors used this model to examine the promotion of hemostasis by prethrombin-1. It was surprisingly shown in this model that prethrombin-1 has an excellent hemostatic effect, as demonstrated in the following example.

Prethrombin-2 has no hemostatic effect in vivo.

The invention is therefore based, among other things, on the realization that prethrombin-1 promotes hemostasis in vivo.

Prethrombin-1 can also be used in particular for the following types of hemorrhage that are difficult to control:
severe acute hemorrhage in patients with coagulation disorders
acute life-threatening hemorrhage resulting from trauma or internal injuries
reversal of anticoagulation under therapies with direct-acting, orally active anticoagulants (NOAKs) due to life-threatening or uncontrolled hemorrhage. NOAKs (new oral anticoagulants) are anticoagulant and antithrombotic agents. The anticoagulant effect is based on the direct inhibition of blood coagulation factors.
Other critical hemorrhage situations in which activated or non-activated prothrombin complex concentrates or recombinant factor VIIa are applied.

“Severe” types of hemorrhage are understood to be those that cannot be stopped by conventional methods.

Prethrombin-1 should have an effect when applied intravenously as well as subcutaneously.

The invention is described in more detail by way of the following examples.

### Example 1: Preparation of prethrombin-1

1 ml of a prothrombin concentrate with an activity of 425 U prothrombin/ml was mixed with 50 U thrombin. The mixture was incubated at room temperature for about 24 hours. After incubation, the mixture was diluted to 10 ml with the buffer solution of 10 mM citrate, 137 mM NaCl, pH 7.0 and applied to 15 ml Heparin Sepharose FF. Prethrombin-1 was isolated from the fraction of the 2^{nd} UV 280 nm (18 ml) signal peak of an isocratic elution with the buffer solution of 15 mM citrate, 150 mM NaCl, pH 7.0, concentrated to 5 ml via a 5 kDa UF membrane, diluted to 10 ml with distilled water and applied to 10 ml AIEX CaptoQ ImpRes. Prethrombin-1 was isolated from the fraction of the 2^{nd} UV 280 nm (22 ml) signal peak of an isocratic elution with the buffer solution of 15 mM citrate, 150 mM NaCl, pH 7.0. The prethrombin-1 fraction was concentrated to 1 ml again via a 5 kDa UF membrane. The prethrombin-1 thus obtained was adjusted to a protein concentration of 5.0 mg/ml.

The prethrombin-1 purified from bands, which had been produced in this way, was formulated with a suitable pharmaceutically acceptable vehicle and formulated into a drug using methods known per se. The prethrombin-1 produced in this way only had about 1% of the activity of prothrombin in the prothrombin assay with prothrombin-deficient plasma. With a complex made of factor Xa and its cofactor Va, one unit of the obtained prethrombin-1 could form about 200 NIH units of thrombin.

### Example 2: Determination of the hemostatic effect in an FVIII inhibitor mouse model

FVB mice were anesthetized and treated with an FVIII antibody via the tail vein in such a way that they showed an increased tendency to bleed after a tail cut. Upon application of the test substance via the femoral vein, the tip of the tail was amputated with a scalpel at a distance of 3 mm. The blood loss was determined gravimetrically. The time that elapsed until the bleeding stopped was indicated as the bleeding time. After the stop of the bleeding, a slight secondary bleeding could occur. The test duration was 30 minutes in all cases.

The results obtained are clearly displayed in the tables below, with the abbreviation “AK” representing the antibody against factor VIII.

Placebo NaCl 0.9%

| | | | | | Bleeding | Measured |
|---|---|---|---|---|---|---|
| | Weight | AK | | NaCl | time | blood loss |
| Name | [g] | [μl] | BU/kg | [μl] | [min] | [mg] |
| MH300 | 23.7 | 40 | 31472 | 144 | >30 | 504.4 |
| MH301 | 23.8 | 40 | 31339 | 144 | >30 | 603.0 |
| MH302 | 24.3 | 40 | 30695 | 144 | >30 | 424.8 |
| MH303 | 24.7 | 40 | 30198 | 144 | >30 | 823.6 |
| MH304 | 24.6 | 40 | 30320 | 144 | >30 | 740.3 |

Prethrombin-1 (57.3 U/ml) 100 U/kg

| | | | | | | | | Mea- |
|---|---|---|---|---|---|---|---|---|
| | | | | | | | | sured |
| | | | | | | Bleeding | | blood |
| | Weight | AK | | Pre-1 | Pre-1 | time | | loss |
| Name | [g] | [μl] | BU/kg | [μl] | U/kg | [min] | | [mg] |
| MH626 | 28.4 | 40 | 24749 | 48.9 | 100 | 6 | min | 0 |
| MH630 | 26.1 | 40 | 26930 | 45.1 | 100 | 9 | min* | 3.7 |
| MH631 | 29.2 | 40 | 24071 | 50.6 | 100 | >30 | min | 0 |
| MH646 | 27.7 | 40 | 25375 | 47.4 | 100 | 2 | min | 0 |

| | | | | | | | |
|---|---|---|---|---|---|---|---|
| MH648 | 25.7 | 40 | 27349 | 45.4 | 100 | 1 min 30 s* | 10.1 |

Prethrombin-1 (57.3 U/ml) 300 U/kg

| | | | | | | | Mea- |
|---|---|---|---|---|---|---|---|
| | | | | | | | sured |
| | | | | | | Bleeding | blood |
| | Weight | AK | | Pre-1 | Pre-1 | time | loss |
| Name | [g] | [μl] | BU/kg | [μl] | U/kg | [min] | [mg] |
| MH633 | 28.1 | 40 | 25014 | 146.6 | 300 | 5 min 10 s* | 51.4 |
| MH634 | 26.2 | 40 | 26827 | 136.1 | 300 | >30 min | 0 |
| MH643 | 27.6 | 40 | 25467 | 146.6 | 300 | 4 min 30 s* | 0 |
| MH644 | 26 | 40 | 27034 | 136.1 | 300 | 4 min 30 s | 0 |
| MH647 | 22.3 | 35 | 27579 | 115.2 | 300 | 1 min 30 s* | 0 |
| MH664 | 26 | 40 | 27034 | 136.1 | 300 | 1 min 40 s* | 0 |

* . . . minor secondary bleeding

The tables show the following:

The placebo group, in which five animals received an isotonic saline solution, had bleeding times>30 minutes, which were associated with blood losses of 424.8 to 823.6 mg.

When prethrombin-1 was administered at a dose of 100 U/kg body weight, the bleeding time in four animals was shortened to 6 minutes, 9 minutes, 2 minutes or 1 minute 30 seconds. Only in one animal, the bleeding time was longer than 30 minutes with very slight bleeding. The blood loss was reduced significantly in all animals, wherein no blood loss at all was measurable in three animals, ended up being 3.7 mg in one animal and ended up being 10.1 mg in another one.

When the prethrombin-1 dose was increased to 300 mg/kg body weight, the bleeding times were 5 minutes 10 seconds, >30 minutes, 4 minutes 30 seconds, 1 minute 30 seconds and 1 minute 40 seconds. Also in this case, the hemorrhage was extremely weak in comparison to the placebo group. No blood loss at all was measurable in 5 animals, and it was 51.4 mg in one animal.

The experiments show that, both with a prethrombin-1 dose of 100 mg/kg body weight and with a dose of 300 mg/kg body weight, the bleeding characteristics were significantly diminished in comparison to the placebo group and, above all, the blood loss could be reduced significantly in comparison to the placebo group.

### Example 3: Thrombogenic potential of prethrombin-1 in a modified Wessler test

The Wessler test has been used for many years to determine the thrombogenicity of various substances in an in-vivo model (27, 28).

White New Zealand rabbits (body weight of 2.5-3.5 kg, Charles River, Germany) were used. The animals were anesthetized and a venous access was established in an ear vein. Thereupon, the contralateral vena jugularis communis was prepared, and the animals were put into a temporary hemophilic status with a factor VIII antibody.

After intravenous application of the test substance, a piece of vein was ligated at a length of 1.5 cm. After a waiting period of 20 minutes, the ligated piece of vein was removed and cut open in an isotonic sodium citrate buffer solution. The luminal vein surface was macroscopically inspected and thrombi, if present, were removed and weighed. The following scale was used for evaluating the thrombi:
0 no thrombus
1 small thrombi weighing<2 mg
2 one or several thrombi
3 a thrombus completely filling the vein

| | | | Wessler |
|---|---|---|---|
| Rabbit No. | Substance | Dose | weighting |
| 329 | prethrombin-1 | 300 U/kg body weight | 0 (no thrombus) |
| 345 | prethrombin-1 | 300 U/kg body weight | 0 (no thrombus) |

The results show that prethrombin-1 is not thrombogenic even at high doses in the Wessler test.

### Example 4: Efficacy of prethrombin-1 in reversing the anticoagulation of oral Xa inhibitors

The efficacy of prethrombin-1 in reversing the anticoagulation of oral Xa inhibitors was demonstrated in a rivaroxaban rabbit model. In doing so, anesthetized New Zealand White rabbits were treated with the factor Xa inhibitor rivaroxaban to achieve anticoagulation. The increased tendency to bleed associated with anticoagulation was determined by cutting the claws. Upon intravenous administration of prethrombin-1, the blood loss was measured again after the claws had been cut.

The results obtained are summarized in the table below.

| | | Blood loss | Prethrombin-1 | Blood loss |
|---|---|---|---|---|
| | Rivaroxaban | after | dose | after |
| Rabbit | dose [μg/kg | rivaroxaban | [units/kg body | prethrombin-1 |
| No. | body weight] | [mg] | weight] | [mg] |
| 350 | 7 | 22.3 | 70 | 0 |
| 353 | 14 | 11.3 | 70 | 0 |
| 355 | 35 | 30.4 | 70 | 20.3 |
| 356 | 70 | 108.5 | 70 | 27.2 |
| 357 | 70 | 51.2 | 70 | 0 |
| 360 | 70 | 165.0 | 70 | 0 |
| 361 | 70 | 98.7 | 70 | 0 |
| 362 | 70 | 20.8 | 70 | 0 |
| 363 | 70 | 51.8 | 23 | 0 |
| 363 | 70 | 77.4 | 70 | 0 |

The dose of rivaroxaban was increased from 7 to 70 mg/kg body weight. Since a sufficient tendency to bleed was achieved at a dose of 70 mg/kg body weight, no further dose increase was necessary. Upon intravenous administration of prethrombin-1, no blood loss was measurable in 10 animals. Blood loss was measurable in two animals, but it was less than after anticoagulation.

It can be concluded from those measurements that prethrombin-1 can reverse the bleeding-promoting effect of oral Xa inhibitors when reversal of anticoagulation is necessary because of life-threatening or uncontrolled hemorrhage.

### LITERATURE

(1) Orfeo T, Butenas S, Brummel-Ziedins K E, Mann K G. The tissue factor requirement in blood coagulation. J Biol Chem. 2005; 280 (52): 42887-96.
(2) Petrillo T, Ayombil F, Van′t Veer C, Camire R M. Regulation of factor V and factor V-short by TFPIa: Relationship between B-domain proteolysis and binding. J Biol Chem. 2021 January-Jun; 296:100234.
(3) Eibl H, Schwarz O and Elsinger F. Patent A T 350726 (1976) Verfahren zur Herstellung einer blutgerinnungsfördernden Präparation aus menschlichem Blutplasma
(4) Eibl H, Schwarz O and Elsinger F. Patent A T 350726 U.S. Pat. No. 4,160,025 (1979) Method of producing a blood-coagulation-promoting preparation from human blood plasma.
(5) Turecek P. and Schwarz H P. (2013) “Factor eight inhibitor bypassing activity” in Production of Plasma Proteins for Therapeutic use, publisher by John Wiley & Sons inc.
(6) Butenas S, van′t Veer C, Mann K G “Normal” thrombin generation. Blood. 1999 Oct. 1;94 (7): 2169-78.
(7) Hansson K M, Lindblom A, Elg M, Lövgren A. Recombinant human prothrombin (MEDI8111) prevents bleeding in haemophilia A and B mice. Haemophilia. 2016 May;22 (3): 453-61.
(8) To Assess Safety, Tolerability and Pharmacodynamics of Intravenous MEDI8111 After Single Ascending Doses.-Study Results-ClinicalTrials.gov
(9) Stojanovski B M, Di Cera E. Role of sequence and position of the cleavage sites in prothrombin activation. J Biol Chem. 2021 August; 297 (2): 100955.
(10) Mann K G, Elion J, Butkowski R J, Downing M, Nesheim M E. Prothrombin. Methods Enzymol. 1981;80 Pt C: 286-302.
(11) Haynes L M, Bouchard B A, Tracy P B, Mann K G. Prothrombin activation by platelet-associated prothrombinase proceeds through the prethrombin-2 pathway via a concerted mechanism. J Biol Chem. 2012 Nov. 9;287 (46): 38647-55.
(12) Chen Z, Pelc L A, Di Cera E. Crystal structure of prethrombin-1. Proc Natl Acad Sci USA. 2010 Nov. 9;107 (45): 19278-83.
(13) Bukys M A, Orban T, Kim P Y, Nesheim M E, Kalafatis M. The interaction of fragment 1 of prothrombin with the membrane surface is a prerequisite for optimum expression of factor Va cofactor activity within prothrombinase. Thromb Haemost. 2008 March;99 (3): 511-22
(14) Ayombil F, Woodet al. Prethrombin-1, the Gla-domainless prothrombin intermediate, is activated efficiently to thrombin by prothrombinase assembled on the activated platelet surface. July 2011. Conference: International Society of Thrombosis and Haemostasis. Japan Volume: Journal of Thrombosis and Haemostasis 9, 352-35
(15) Friedmann A P, Koutychenko A, Wu C, Fredenburgh J C, Weitz J I, Gross P L, Xu P, Ni F, Kim P Y. Identification and characterization of a factor Va-binding site on human prothrombin fragment 2. Sci Rep. 2019 Feb. 21;9 (1): 2436.
(16) McDuffie F C, Giffin C, Niedringhaus R, Mann K G, Owen C A Jr, Bowie E J, Peterson J, Clark G, Hunder G G. Prothrombin, thrombin, and prothrombin fragments in plasma of normal individuals and of patients with laboratory evidence of disseminated intravascular coagulation. Thromb Res. 1979;16 (5-6): 759-73.
(17) Owen C A Jr, Mann K G, McDuffie F C. The turnover in normal dogs of prothrombin and its fragments; effect of induced intravascular coagulation. Thromb Haemost. 1979 Aug. 31;42 (2): 548-55.
(18) Lanchantin G F, Friedmann J A, Hart D W. On the occurrence of polymorphic human prothrombin. Electrophoretic and chromatographic alterations of the molecule due to the action of thrombin. Biol Chem. 1968 Feb. 10;243 (3): 476-86.
(19) Heldebrant C M, Butkowski R J, Bajaj S P, Mann K G The activation of prothrombin. II. Partial reactions, physical and chemical characterization of the intermediates of activation. J Biol Chem. 1973 Oct. 25;248 (20): 7149-63.
(20) Seegers W H, Hassouna H I, Novoa E. Immunological aspects of some vitamin K-dependent factors and preparation of depleted plasmas. Thromb Res. 1977 November;11 (5): 633-42.
(21) Baker W J, Seegers W H. The conversion of prethrombin to thrombin. Thromb Diath Haemorrh. 1967 Feb. 28;17 (1-2): 205-13.
(22) Seegers W H, Ghosh A. Activation of prothrombin and factor X: function of previously unrecognized plasma protein. Thromb Res. 1980 Jan. 1-15;17 (1-2): 71-81.
(23) Seegers W H, Hassouna H I, Novoa E. Immunological aspects of some vitamin K-dependent factors and preparation of depleted plasmas. Thromb Res. 1977 November;11 (5): 633-42.
(24) Meehan W P et al. Recombinant human thrombin: Demonstration of efficacy in models of surgical bleedings. Journal of surgical research (2004 Oct. 1) 323
(25) Stroethmann M EP 0 068 048 A2
(26) Varadi K, Tangada S, Loeschberger M, Montsch P, Schrenk G, Ewenstein B, Turecek P L. Pro- and anticoagulant factors facilitate thrombin generation and balance the haemostatic response to FEIBA® in prophylactic therapy. Haemophilia. 2016 July;22 (4): 615-24.
(27) S. Wessler, S. M. Reimer, M. C. Sheps Biologic assay of a thrombosis-inducing activity in human serum J Appl Physiol, 14 (1959), pp. 943-946
(28) A. R. Giles, M. Johnston, H. Hoogendoorn, M. Blajchman, J. Hirsh The thrombogenicity of prothrombin complex concentrates: I. The relationship between in vitro characteristics and in vivo thrombogenicity in rabbits Thromb Res, 17 (1980), pp. 353-366

1-8. (canceled)

9. A method of treating a disease or condition comprising administering to a subject in need thereof a therapeutically effective amount of prethrombin-1 to treat the disease or condition.

10. The method of claim 9, wherein administering the prethrombin-1 to the subject promotes hemostasis.

11. The method of claim 9, wherein administering the prethrombin-1 to the subject treats a hemorrhage.

12. The method of claim 11, wherein administering the prethrombin-1 to the subject treats a hemorrhage associated with a coagulation disorder.

13. The method of claim 11, wherein administering the prethrombin-1 to the subject treats a hemorrhage resulting from trauma or internal injuries.

14. The method of claim 11, wherein administering the prethrombin-1 to the subject treats a hemorrhage associated with breakthrough bleeds under substitution treatments with coagulation factors, therapy mimicking factor VIII, or gene therapy.

15. The method of claim 9, wherein administering the prethrombin-1 to the subject reverses anticoagulation by direct-acting anticoagulants administered orally.

16. The method of claim 9, wherein the prethrombin-1 is recombinant prethrombin-1.

17. A method of promoting hemostasis comprising administering to a subject in need thereof a therapeutically effective amount of prethrombin-1 to promote hemostasis.

18. The method of claim 17, wherein administering the prethrombin-1 to the subject treats a hemorrhage.

19. The method of claim 18, wherein the hemorrhage is associated with a coagulation disorder.

20. The method of claim 18, wherein the hemorrhage results from trauma or internal injuries.

21. The method of claim 18, wherein the hemorrhage ius associated with breakthrough bleeds under substitution treatments with coagulation factors, therapy mimicking factor VIII, or gene therapy.

22. The method of claim 17, wherein administering the prethrombin-1 to the subject reverses anticoagulation by direct-acting anticoagulants administered orally.

23. The method of claim 17, where the prethrombin-1 is recombinant prethrombin-1.
